(19) United States
(12) Patent Application Publication
(10) Pub. No.: US 2006/0088818 A1
(43) Pub. Date: Apr. 27, 2006

(54) Optical detection of microorganisms and toxins

(76) Inventors: William Douglas Beynon, Waterloo (CA); Mike Jackson, Winnipeg (CA)

Correspondence Address: ADE & COMPANY INC., P.O. BOX 28006 1795 HENDERSON HIGHWAY, WINNIPEG, MB, R2G1P0, CA

(21) Appl. No.: 11/055,720

(22) Filed: Feb. 11, 2005

Related U.S. Application Data
(60) Provisional application No. 60/543,272, filed on Feb. 11, 2004.

(51) Int. Cl. C12Q 1/70; G01N 33/554

(52) U.S. Cl. 435/5; 435/7.32

(57) ABSTRACT

A method of detecting the presence of selected microorganisms within a fluid includes filtering the fluid to remove large particles prior to analyzing the fluid with an antibody matrix. Non-specific binding is eliminated by washing and the presence of biological material is detected. If biological material is detected within the matrix, specific secondary antibodies are added which confirm the presence of the microorganism of interest and are also used to quantitate the level of the microorganism within the sample.

### PRIOR APPLICATION INFORMATION

This application claims priority on U.S. Provisional Patent Application 60/543,272, filed Feb. 11, 2004.

### FIELD OF THE INVENTION

The present invention relates generally to the field of spectroscopy and spectroscopic imaging. More specifically, the present invention relates to a method for continuous, real time monitoring of air and water to detect biological warfare agents using a variety of optical techniques.

### BACKGROUND OF THE INVENTION

It is becoming increasingly likely that terrorists will use biological weapons in attacks on Western countries. Anthrax, plague and smallpox have been identified as agents of particular concern. Probable scenarios for bioterrorism (BT) events include release of aerosolized BT agents in a public place such as a sports arena or shopping mall or by more general mechanisms such as crop spraying planes. Currently, a BT event could only be detected when patients present clinical symptoms. Clearly for highly communicable diseases such as smallpox this is unacceptable, because by the time symptomatic patients appeared in hospitals, the disease would have spread across the North American continent. Clearly, methods are needed to detect a BT event as it happens so that appropriate action (quarantine, decontamination, vaccination, transport of therapeutics) can be initiated. Detection of such events requires continuous, real time, unattended monitoring of air. There is no system currently available that allows this to be done.

Detection and identification of microorganisms is also required for less dramatic but equally important scenarios, such as monitoring air quality in buildings to reduce so-called “sick building syndrome” and monitoring the quality of water in lakes, food and beverage processing and/or handling and water treatment facilities. Detection and identification of microorganisms in such situations may prevent the spread of agents such as those responsible for Legionnaires Disease, typhoid and cholera, as well as less exotic but equally important organisms such as E. coli and Cryptosporidium.

Thus, a rapid, continuous monitoring technique that could be utilized for assessment of BT agents in air and water would be valuable tool for both civilian and military defence.

Specific detection or localization of a number of analytical materials (typically proteins) may be achieved using the technique of immunoassay. Such assays are based upon the specific interaction between an antibody and the corresponding antigen. Localization or detection of the bound antibody, and by inference the antigen, is usually achieved by optical, enzymatic or radiation-based techniques such as fluorescence, chemiluminescence, electroluminescence and beta/gamma emission.

In addition to being useful for identification and localization of specific molecules, immunoassays can also be used to detect intact cells. If the cell of interest expresses an antigen that is accessible to an appropriate antibody, then incubation of a suspension of cells with the antibody will result in binding of the antibody to cells expressing the antigen. The use of an optically labelled antibody will then allow detection of the presence of the cell of interest. We make use of the specific interaction between antibodies generated to biological warfare agents and the agent in question to develop a device capable of detecting low levels of biowarfare agents in air and/or water.

### SUMMARY OF THE INVENTION

The device comprises an air/water sampling unit which concentrates particulate matter onto a support; a main analyser unit which contains a matrix to selectively trap bacteria, viruses and toxins of interest, and the required reagents; and an optical or other type of sensing system. The mode of operation is summarized in the flow chart shown in FIG. 1.

According to the invention, there is provided a method for detecting a microorganism in a fluid comprising:

a) providing a sample of a fluid to be analyzed;

b) filtering the sample;

c) passing the sample over a plurality of primary antibodies under conditions suitable for antibody binding, a respective one of said plurality of primary antibodies specifically binding an antigen for a microorganism of interest; and

d) detecting the presence of biological material specifically bound at at least one of said respective antibodies, wherein a positive signal indicates the presence of at least one microorganism of interest.

Each respective one of the primary antibodies may be covalently linked to a functionalised support.

If the fluid is air, the method includes, following step (b),

b1) passing the sample over an impactor, said impactor binding particles within the sample; and

b2) washing the impactor with a buffer.

The plurality of primary antibodies may be biotinylated and attached to a support with avidin or streptavidin.

The plurality of primary antibodies may be labelled.

The label may be selected from the group consisting of a substrate suitable for Surface Enhanced Raman Spectroscopy (SERS), a fluorescent label, a chemiluminescent label, an electroluminescent label, an enzyme-antiboy construct, a polymerized enzyme antibody construct and other similar suitable labels known in the art.

The presence of biological material bound to the primary antibodies may be detected by a fluorescence signal generated due to the presence of NADH, tyrosine, or tryptophan, thereby indicating the presence of at least one microorganism of interest. Following the detection of biological material bound to the primary antibodies, labelled secondary antibodies directed against said microorganism of interest may be added to the sample and the amount of bound secondary antibodies may be measured.

Alternatively, the presence of biological material may be detected by adding labelled secondary antibodies directed against to the sample and detecting bound secondary antibodies.

The primary antibodies may be labelled and signal generated from the primary antibody and secondary antibody may be detected by optical imaging using an array of detectors.

The signal generated from the primary antibody and secondary antibody may be imaged by scanning the analyser using a single detector element.

The signal generated from the primary antibody and secondary antibody may be detected with a fixed (non-scanning) optical system by serially uncovering each cell in the analyser.

The signal generated from the primary antibody and secondary antibody may be detected with a fixed (non-scanning) optical system by positioning of a single detector element in front of or behind each cell in the analyser.

The signal generated from the primary antibody and secondary antibody may be detected electrically or electrochemically.

The proportion of available binding sites occupied may be calculated from the ratio of the signals generated from the primary and secondary antibodies.

### BRIEF DESCRIPTION OF THE DRAWINGS

FIG. 1 shows a flow chart outlining one embodiment of the mode of operation of the device.

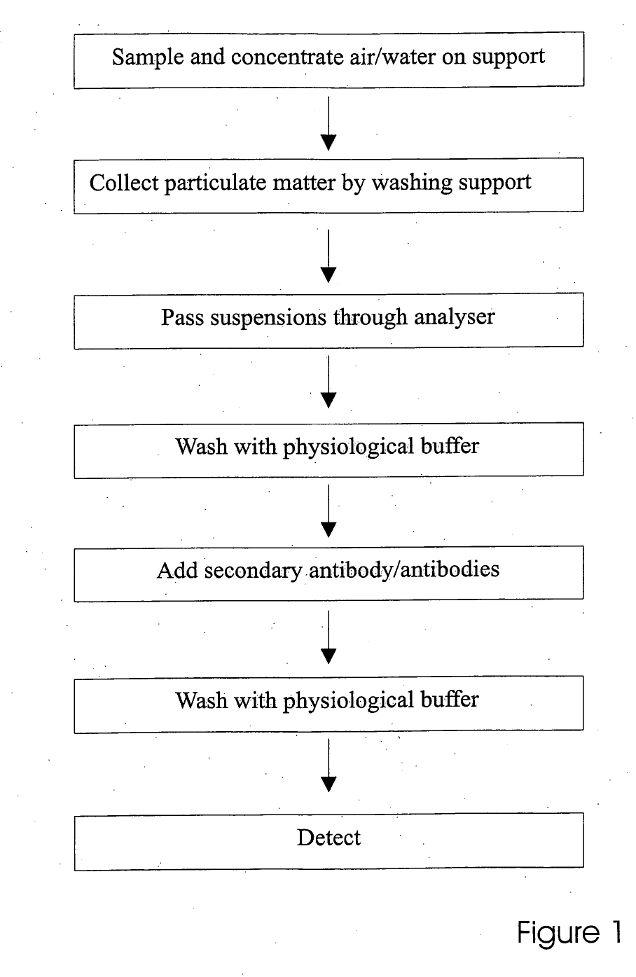

FIG. 2 shows one embodiment of the matrix used to trap BT agents.

FIG. 3 shows a schematic representation of one embodiment of a device for trapping and detection of BT agents using an immunofluorescence imaging approach,

FIG. 4 shows the result that would be obtained if immunofluorescence imaging was used to assess the distribution of a primary (trapping) antibody within the matrix.

FIG. 5 shows the result that would be obtained if immunofluorescence imaging was used to assess the distribution of a secondary (detection) antibody to anthrax within the matrix if the matrix is exposed to anthrax.

FIG. 6 shows the result that would be obtained if immunofluorescence imaging was used to assess the distribution of a secondary (detection) antibody to smallpox within the matrix if the matrix is exposed to smallpox.

FIG. 7 shows the result that would be obtained if immunofluorescence imaging was used to assess the distribution of secondary (detection) antibodies to anthrax and smallpox within the matrix if the matrix is exposed to both anthrax and smallpox.

FIG. 8 shows one scheme for producing an intense, continuously generated luminescence signal using biotinylated antibodies and an avidin-enzyme construct.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Definitions

As used herein, “Immunoassay” refers to a test using antibodies to identify and quantify substances. Often the antibody is linked to a marker such as a fluorescent molecule, a radioactive molecule, or an enzyme.

As used herein, “Fluorescence” refers to the emission of light at one wavelength following absorption of light with a shorter wavelength.

As used herein, “Luminescence” refers to the emission of light stimulated by chemical or electrical means.

As used herein, “Optical detection” refers to detection of species of interest using light. Such detection may be based upon absorption or emission of light.

As used herein, “microorganism” refers to for example, fungi, bacteria, spores thereof, and viruses.

Described herein is a method of detecting microorganisms in a fluid. As discussed above, the microorgansims may be bacteria, fungi, spores thereof, viruses or the like. In a preferred embodiment, the microorganisms are organisms associated with bioterror and/or bioweapons, for example, but by no means limited to, smallpox virus, anthrax, plague and the like. The fluid may be air or a liquid such as water. It is noted that in a preferred embodiment, the method samples a fluid from the environment, for example, air or drinking water which is in contrast with methods arranged for the analysis of bodily fluids such as blood, saliva and the like. As such, in a preferred embodiment of the invention, the fluid is a non-bodily fluid.

In the described method, the fluid is first filtered to remove large particles. In a preferred embodiment, discussed below, when the fluid is air, a commercially available filter such as a HEPA filter is used to filter out large particles. If the fluid is air, the filtered fluid is then passed over an impactor which traps all particles within a certain size range that impact upon it. At regular intervals which are determined by the user, the impactor is washed for example with a buffer to remove any particles which are then passed on to the analyzer. If the fluid is water, a water filter is used to remove large particles and the water sample is passed directly to the analyzer, as discussed below.

The analyzer includes a sensor which comprises a plurality of antibodies in a matrix and the wash is passed over these antibodies such that any material expressing or presenting an epitope or region recognized by an antibody within the antibody matrix is specifically retained. The matrix is then washed again to remove any non-specifically retained material from the impactor wash. The antibody matrix is then screened for the presence of one or more signals indicative of the presence of microorganisms. In one embodiment, this optical trigger may be based upon fluorescence of tyrosine, tryptophan and/or NADH, as discussed below. In a preferred embodiment, the antibody matrix is ordered or sortable such that the presence of a signal at a given location indicates binding at or by a specific antibody or antibody class. Specific labelled secondary antibodies are then released into the antibody matrix which both confirm the presence of the antigen or epitope and are also used to quantitate the amount of antigen and by extension microorganism of interest within the sample, as discussed below.

Configuration for Real Time Monitoring of Air

Air Sampling

Air is continuously sampled by the analyser at an adjustable flow rate. The inflowing air will be screened (for example using HEPA filters or other size-specific sampling technology) to remove large particles that may interfere with the analysis (for example but by no means limited to mould, fungus, dust particles, pollen etc). It is noted that HEPA filters and the like are well-known in the art and the size of particles excluded by a HEPA filter and the similar filters is well-known. The filtered air is captured on an impactor that traps all particles impinging upon it within a predefined size limit. The impactor may be made of any suitable material, for example but by no means limited to polyurethane foam. It is noted that other suitable materials which will preferably retain materials within a specific size range are well known in the art and may be used within the invention. Following a predetermined sampling time, the support material is washed with a buffer, for example, a physiological buffer, that is, a suitable buffer that substantially preserves the native state, that is, does not significantly denature, the material to be sampled. Examples of such buffers are well known in the art but include for example, water, PBS, Krebs solution, Ringers solution, tris (hydroxymethyl) aminomethane, bicarbonate buffer and the like. That is, any suitable buffer known in the art for antibody-antigen interactions, dilution of viruses or bacteria, maintenance of bacterial or viral stocks and the like. It is of note that the physiological buffer will also be suitable for antibody-antigen binding, as discussed below. The buffer and any suspended materials (including viral particles, bacteria cells and toxins) are then passed into the sensor.

Microbial and Toxin Sensor Design

The microbial and toxin sensor is based upon a sandwich immunoassay assay using optical or some other type of detection. In a general configuration, an antibody (the primary antibody) is attached to a surface (support structure) and when the respective biological material is passed over the surface, it attaches to the antibody. All other materials are removed by washing with physiological buffer. The system is then incubated with a buffer containing an antibody to the suspected agent (secondary antibody, which may be the same antibody as that bound to the support or a different antibody specific to the same BT agent). The primary and secondary antibodies are labeled with different fluorescent dyes (for example Cy5 and Cy7) that allow optical determination of the amount of primary and secondary antibody present. Detection of the secondary antibody confirms the presence of the suspected BT agent, while the secondary/primary antibody fluorescence ratio allows an estimation of the relative concentration of BT present. The sensor may be implemented in at least two support configurations, with each configuration allowing a user determined number of species of interest to be identified.

Support Configuration 1.

Configuration 1 uses functionalised beads as the support for the sandwich immunoassay. The key property of beads is the large surface area provided by the spherical shape. By packing beads in a column or compartment, a very large surface area can be achieved. In this configuration, a monoclonal antibody (the primary monoclonal antibody, Mab1a) to a species of interest is covalently attached to functionalised beads. The beads may be of any construction that allows functionalisation (e.g. metallic, organic or mineral such as glass or quartz) and can be of any size. Factors that must be considered when choosing the support include but are not limited to a) ease and reproducibility of functionalisation, b) cost, c) surface area, and d) transmission characteristics. Metal oxide beads present limitations due to complete optical opaqueness, which precludes construction of a device in any configuration but one utilizing illumination and collecting from the same side of the analyser.

To allow estimation of the quantity of antibody attached to the beads, the antibody is coupled to an optical marker (for example but by no means limited to a fluorescent dye such as Cy5, Cy7 etc. or a luminescent probe). This coupling may be performed before or after the antibody is attached to the bead.

To allow simultaneous determination of two or more species (or to perform strain typing), multiple monoclonal antibodies (Mab1a, Mab2a, Mab3a etc) can be attached to the same beads. To allow quantitation of each antibody, each antibody should be labeled with a separate optical marker. However, the number of useful optical markers is limited. It is therefore more feasible, for detection of more than 3 species, to attach each monoclonal antibody to a separate population of beads. In this embodiment, each antibody can then be labeled with the same optical marker.

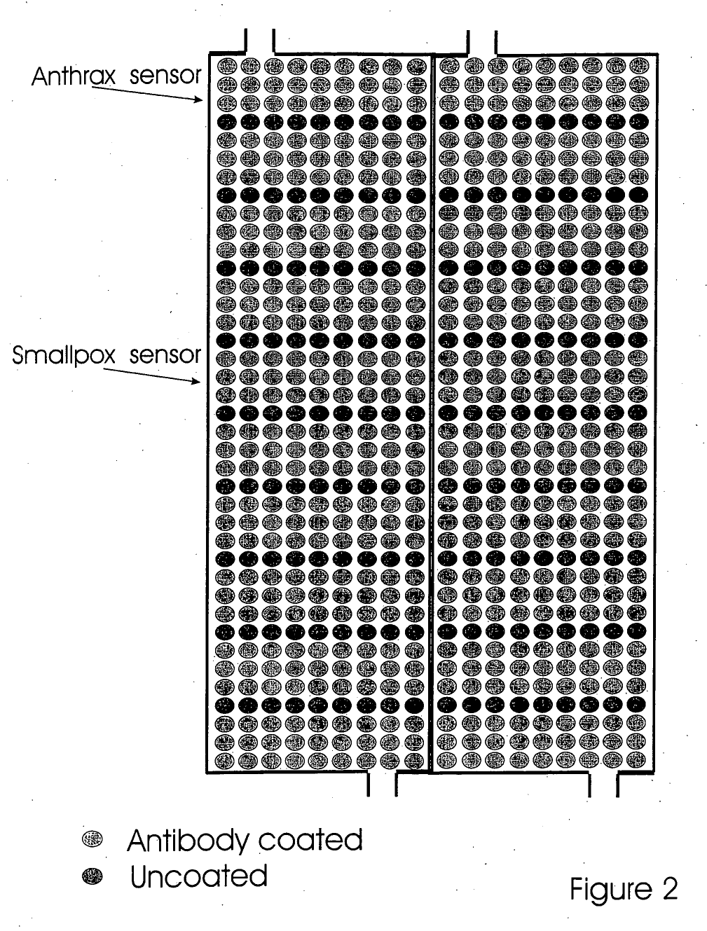

The sensor is constructed by packing either single or multi-antibody labeled beads in a single column, or for determination of a large numbers of species, by packing individually labeled beads in spatially distinct compartments (see FIG. 2). In the design exemplified in FIG. 2, each compartment is delineated by non-functionalised beads and contains beads coupled to antibodies for a different virus, bacterium or toxin. This embodiment provides a large surface area for antibody attachment, thereby increasing sensitivity.

An alternative mode of coupling antibody to the support surface may use the high affinity and specificity of the interaction between the water-soluble vitamin biotin and the proteins streptavidin or avidin. In this embodiment the support material is coated with avidin or streptavidin. Commercially available streptavidin or avidin coated polystyrene beads are available and can be produced with an extremely high surface density of protein molecules. The primary antibody is covalently labeled with biotin. Biotinylation may be confirmed by treatment of biotinylated antibody with pronase to release free biotin, which can be detected colourimetrically using a HABA/avidin displacement assay. In this assay biotin covalently linked to proteins is released by pronase activity, and the free biotin displaces a substrate (HABA) from avidin.

Incubation of the support material with the labeled antibody results in immobilization of the antibody on the support surface. To allow estimation of the quantity of antibody attached to the beads, the antibody is coupled to an optical marker (for example a fluorescent dye such as Cy5, Cy7 etc. or a luminescent probe). This coupling may be performed before or after the antibody is attached to the bead.

Based upon comparisons of images of streptavidin-biotin-antibody-Cy5 labeled beads with images of solutions of Cy5, the amount of Cy5 detected can be estimated. Approximately the same fluorescence intensity is observed with a 2 second acquisition from 1.93×10⁻¹¹ moles of Cy5 and a 2 minute acquisition of the labeled BSA. This implies the presence of 60 times less or 3.22×10⁻¹³ moles of Cy5 on the beads. Assuming a dye: protein ratio of 3:1 (the average ratio under the coupling conditions used) this translates to approximately 1×10⁻¹³ moles of antibody linked to the beads.

Support Configuration 2.

Configuration 2 uses a flat support for the sandwich immunoassay. Briefly, a monoclonal antibody (Mab1a) to a species of interest is covalently attached to a flat functionalised surface (such as glass, quartz, plastic etc). To allow estimation of the quantity of antibody attached to the support, the antibody is coupled to an optical marker (OM1, for example a fluorescent dye such as Cy5, Cy7 etc. or a luminescent probe). This coupling may be performed before or after the antibody is attached to the support.

To allow simultaneous determination of two or more species (or to perform strain typing) multiple monoclonal antibodies (Mab1a, Mab2a, Mab3a etc) can be attached to the support. To allow quantitation of each antibody, each antibody should be labeled with a separate optical marker. However, the number of useful optical markers is limited. It may therefore be more feasible for detection of more than 3 species to spatially separate each monoclonal antibody. In this embodiment, each antibody can then be labeled with the same optical marker (OM1). Preparation of such a sensor may be achieved with technology commonly used to prepare DNA chips. This embodiment has the advantage that the sensor can easily be mass-produced using low cost materials.

An alternative mode of coupling antibody to the support surface may use the high affinity and specificity of the interaction between the water-soluble vitamin biotin and the proteins streptavidin or avidin. In this embodiment the support material is coated with avidin or streptavidin. The primary antibody is covalently labeled with biotin. Incubation of the support material with the labeled antibody results in immobilization of the antibody on the support surface. To allow estimation of the quantity of antibody attached to the beads the antibody is coupled to an optical marker (for example a fluorescent dye such as Cy5, Cy7 etc. or a luminescent probe). This coupling may be performed before or after the antibody is attached to the bead.

Sensor Operation

In one embodiment, the sensor is arranged to allow simultaneous determination of 20 species (20 sets of beads labeled with 20 monoclonal antibodies, as in FIG. 2, or 20 well defined regions on a planar surface). Following washing of the impactor with physiological buffer, the buffer is passed into the sensor chamber. Materials expressing or presenting antigens recognised by any of the 20 antibodies present in the sensor will be sequestered in the appropriate chamber or bound to the appropriate area on the planer support. Other material will pass through the sensor and be captured on a filter. The buffer regenerated in this manner will be re-circulated through the sensor (to ensure efficient distribution of particulate material though the sensor). Following re-circulation of the buffer through the sensor, the sensor will be washed with physiological buffer to remove non-specifically retained material.

At this point the sensor can operate in a triggered mode or in a continuous mode. In the triggered mode, an optical sensor is used to determine whether or not material has been captured within the sensor. The optical trigger may be based upon fluorescence of tyrosine, tryptophan and/or NADH, although the presence of other similar compounds may also or alternatively be detected. As will be appreciated by one of skill in the art, these compounds are typically found in biological organisms and the presence of this material within the washed sensor would indicate the likely presence of a biological organism of interest. Specifically, the sensor cartridge is illuminated with light at the appropriate wavelengths to stimulate fluorescence of tyrosine, tryptophan and/or NADH. Fluorescence may be sensed using either a single sensing element for example but by no means limited to a photodiode, an avalanche photodiode or a photomultiplier tube or by using an imaging array. If a negative response is received to the optical trigger, then sampling commences again. A positive response from a single sensing element would initiate passage of a series of secondary monoclonal antibodies (Mab1b, Mab2b . . . Mab20b) for each species labeled with a second optical marker (OM2) through the sensor. Secondary monoclonal antibodies will bind to species sequestered by primary antibodies. A positive response from an imaging array would trigger passage of a single secondary monoclonal antibodies (Mabxb) for a particular species (based upon localisation of the signal within in the sensor, see below) labeled with a second optical marker (OM2) through the sensor. The column is then rinsed once more with physiological buffer to remove unbound secondary antibody and an optical detection scheme is used to localise and quantitate OM1 and OM2.

As will be appreciated by one of skill in the art, the optical detection scheme utilised will depend on the optical marker used. In the current embodiment we will assume fluorescence detection, although other embodiments may use other optical detection schemes (such as luminescence detection). We will also assume that the sensor design illustrated in FIG. 2 is employed, which requires only two fluorescent markers.

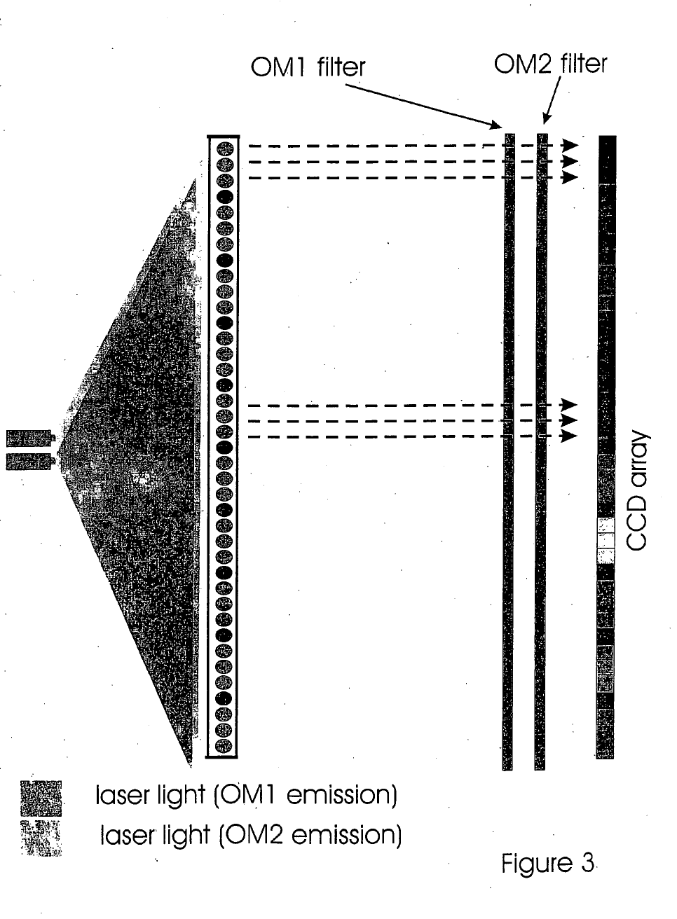

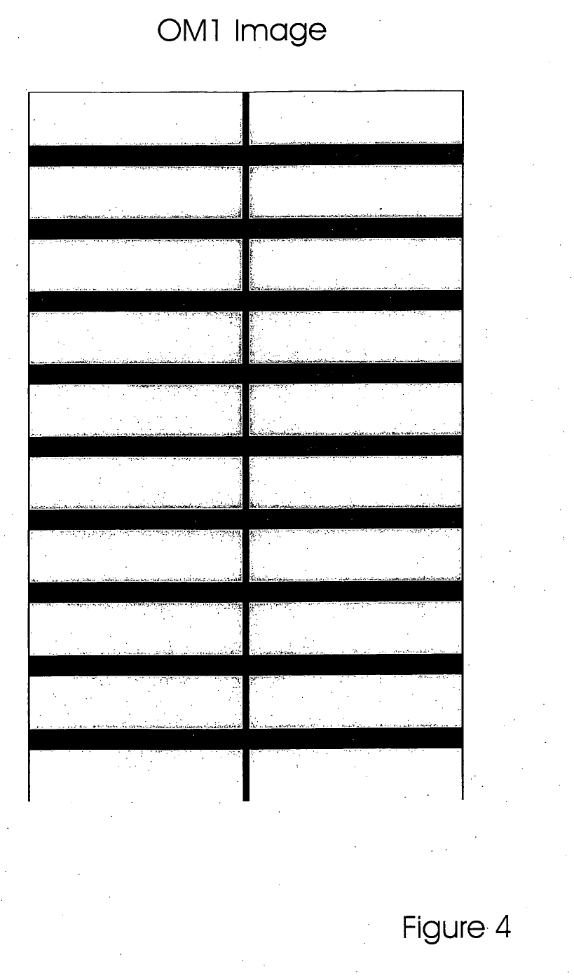

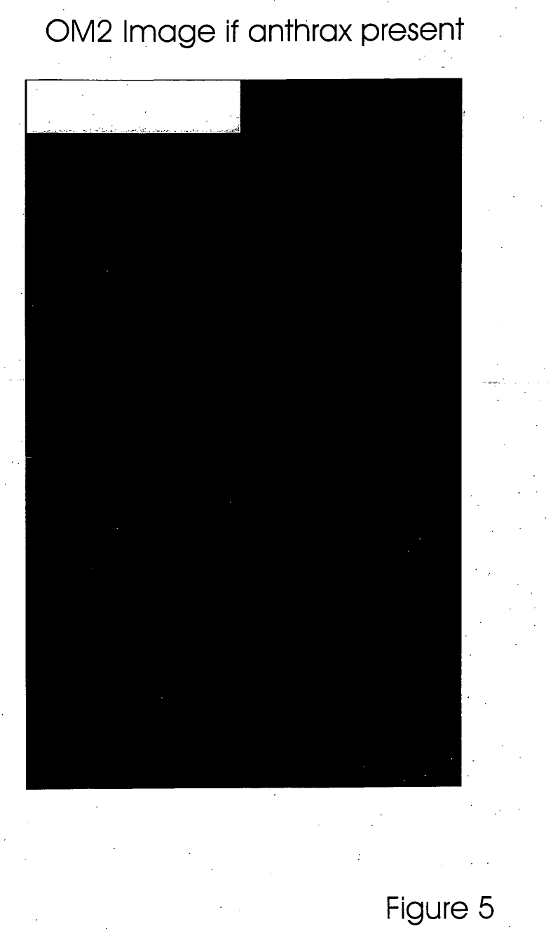

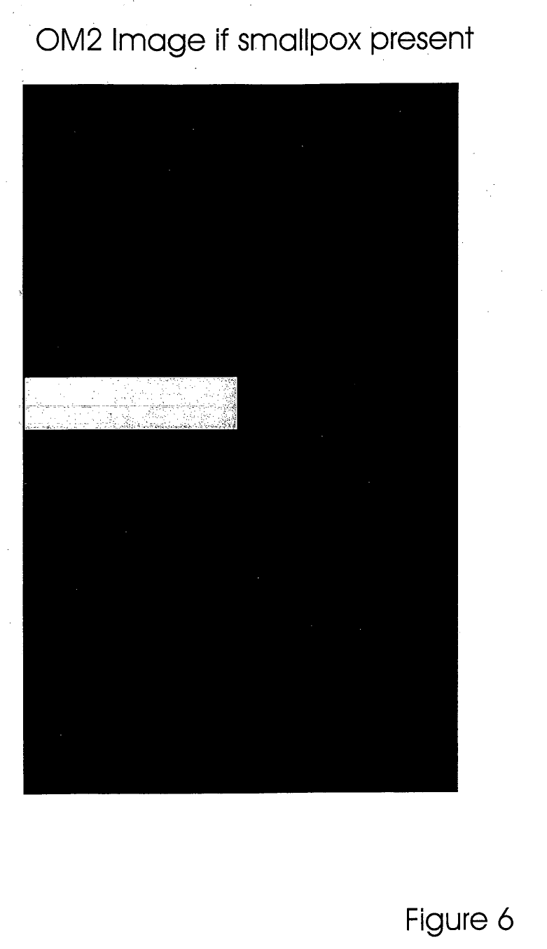

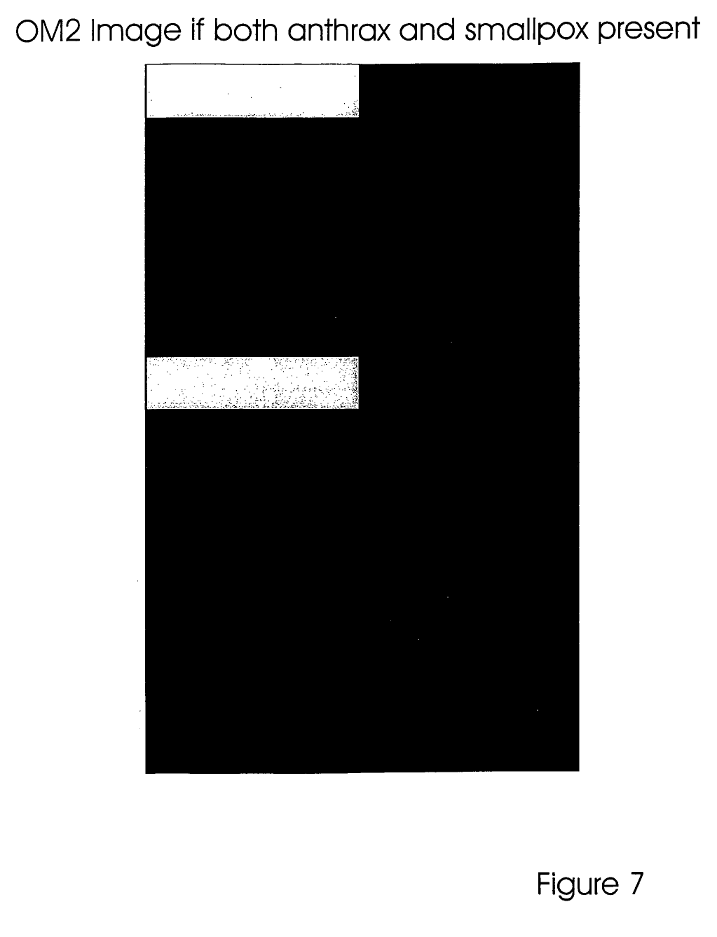

For fluorescence detection the device employs two low power laser diodes at the excitation wavelengths of OM1 and OM2 (FIG. 3). The device is first illuminated at the excitation wavelength of OM1. Fluorescence is imaged using a charge-coupled device array or similar detector equipped with a band pass or other filter designed to optimise fluorescence detection from OM1. As all Mabs are labeled with OM1, then the image obtained will resemble that illustrated in FIG. 4. The sensor is then illuminated at the excitation wavelength of OM2 using a band pass or other filter designed to optimise fluorescence detection from OM2. Fluorescence from OM2 will only be observed in compartments that have sequestered species expressing antigens recognised by Mab1a/b, Mab2a/b, . . . Mab20a/b. For example, if anthrax or smallpox is present, then we expect to see the image illustrated in FIGS. 5 and 6 respectively. If both are present, then the image illustrated in FIG. 7 would be seen. The proportion of binding sites occupied can be estimated by calculating a ratio of the OM2 and OM1 images.

In the continuous mode, optical triggering is not used. Rather, in these embodiments, secondary antibodies are passed into the sensor after each washing of the impactor. Following washing to remove unbound secondary antibodies, the optical detection scheme outlined above is used to localise and quantitate OM1 and OM2.

In another embodiment, a point measurement system is used rather than imaging. Replacing the CCD array detector with a single photodiode detector would give approximately an order of magnitude improvement in sensitivity. The photodiode provided increased sensitivity by eliminating the spacial resolution. The CCD array provides spatial resolution allowing one the ability to determine where in the compartment each signal originates. The photodiode detector concentrates the light on one detector providing better sensitivity, but, no special resolution. Placing a photodiode behind each cell in the analyser could result in a system that would allow detection of 6 million cells. A similar arrangement using avalanche photodiodes would lower the detection limit a further order of magnitude, allowing detection of 600,000 cells, while in principle the use of photomultiplier tubes would allow detection of 60,000 cells in principle. Detection limits could be further improved using single point detection systems due to reductions in the distance between the emitter and sensor compared to an imaging arrangement.

In a further embodiment, an alternative to the use of 20 detectors would be the use of a single high sensitivity detector such as a photomultiplier detector with a parabolic mirror. In this implementation each cell in the analyzer would be equipped with a light-tight window, which would be opened sequentially to allow sampling from each cell in turn. Light from each cell would impinge upon the parabolic mirror, allowing the use of only one detector to serially monitor photons from all 20 cells.

For maximal sensitivity, immuonofluorescence techniques require optimisation of the dye used, protein-dye ratios, illumination-detection geometry, integration time, instrumentation (detection methodology) and of course antigen levels within detection limits. If fluorescence detection is utilized, increased fluorescence yields may be obtained by increasing dye:protein ratios, but increasing the dye:protein ratio above 5-8 may result in loss of activity of antibodies. In addition, fluorescence quenching becomes an issue, potentially reducing yields rather than improving them.

In a yet further embodiment, chemiluminescence detection may be utilised. Many chemiluminescent agents are available, such as acridinium ester. Acridinium ester is commercially available in a form that is readily conjugated to proteins and is readily activated by hydrogen peroxide. Up to 10 acridinium molecules may be attached to antibodies. The use of an appropriate chemiluminescent label will therefore result in at least an order of magnitude improvement in detection limits (due to increased labeling with no quenching and improved efficiency of the light generating process).

A drawback of such chemiluminescent detection schemes is the short lifetime of the chemiluminescent signal (a few seconds). Furthermore, once triggered, the reaction cannot be re-initiated as the luminescent material will have undergone chemical conversion to the non-luminscent form.

In another embodiment, a more effective method for increasing detection limits is to use a detection system that produces a luminescent signal through an enzymatic process. Such a scheme would require a stable, high turnover enzyme that results in luminescence. Alkaline phosphatase (AP) is often used for such detection schemes. This enzyme is available in a maleimide activated form simplifying conjugation to antibodies. In the presence of substrate (such as Lumigen APS-5 from Lumigen Inc.) AP produces light at 450 nm. Light is continually produced as long as substrate is available, allowing long integration times and repeated probing, in contrast to standard chemiluminescence probes. Detection of this light allows detection of levels of AP of 10⁻¹⁹ moles or better.

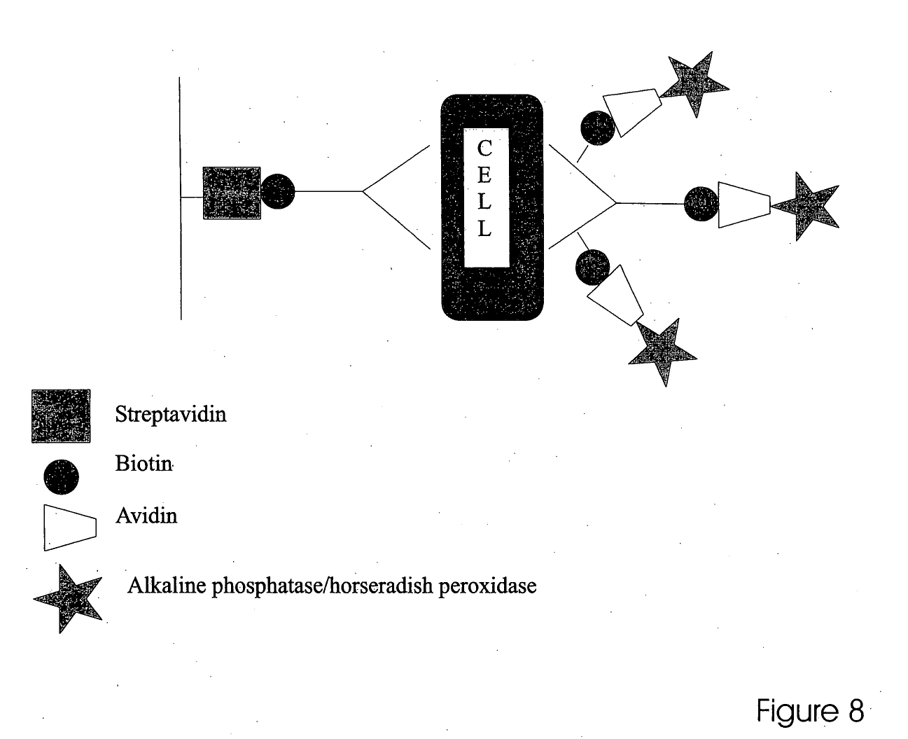

Another embodiment which does not require chemical coupling of the luminescent agent to the secondary antibody is to use a biotinylated secondary antibody and an enzymatic detection system such as avidin-alkaline phosphates or avidin-horseradish peroxidase. In this implementation a streptavidin coated surface is used to attach the primary antibody, which traps the bacterial cell. The support-primary antibody-bacterial cell is then treated with a multi-biotinylated secondary antibody which binds to the bacterial cell. Addition of an avidin-alkaline phosphatase or avidin-horseradish peroxidase construct results in binding of the construct to the biotinylated antibody, producing a highly enzymatically active product (see FIG. 8) capable of producing a luminescent signature. Such a scheme with appropriate high sensitivity detection technology (such as a photomultiplier tube) should be capable of detecting 10⁻¹⁹ moles of alkaline phosphatase, corresponding to 10⁻¹⁹ moles of secondary antibody. Theoretically, this translates into 600,000 secondary antibody molecules, or about 1000 cells. The use of polymerized enzymes, or multiply biotinylated antibodies and an avidin-enzyme construct will further enhance sensitivity (by increasing the ratio of enzyme: antibody).

Configuration for Real Time Monitoring of Water

The configuration for water monitoring is essentially identical to the configuration for air monitoring, with the exception that the initial air filtration step to remove large particles is replaced by a water filtration system and that the water will be transferred directly to the sensor without the use of an impactor.

While the preferred embodiments of the invention have been described above, it will be recognized and understood that various modifications may be made therein, and the appended claims are intended to cover all such modifications which may fall within the spirit and scope of the invention.

1. A method for detecting a microorganism in a fluid comprising:
a) providing a sample of a fluid to be analyzed;
b) filtering the sample;
c) passing the sample over a plurality of primary antibodies under conditions suitable for antibody binding, a respective one of said plurality of primary antibodies specifically binding an antigen for a microorganism of interest; and
d) detecting the presence of biological material specifically bound at at least one of said respective antibodies, wherein a positive signal indicates the presence of at least one microorganism of interest.

2. The method according to claim 1 wherein each respective one of the primary antibodies is covalently linked to a functionalised support.

3. The method according to claim 1 wherein the fluid is air.

4. The method according to claim 3 including, following step (b),
b1) passing the sample over an impactor, said impactor binding particles within the sample; and
b2) washing the impactor with a buffer.

5. The method according to claim 1 wherein the plurality of primary antibodies are biotinylated and attached to a support with avidin or streptavidin.

6. The method according to claim 1 wherein the plurality of primary antibodies are labelled.

7. The method according to claim 6 wherein the label is a substrate suitable for Surface Enhanced Raman Spectroscopy (SERS).

8. The method according to claim 1 wherein the presence of biological material bound to the primary antibodies is detected by a fluorescence signal generated due to the presence of NADH, tyrosine, or tryptophan, thereby indicating the presence of at least one microorganism of interest.

9. The method according to claim 8 wherein following the detection of biological material bound to the primary antibodies, labelled secondary antibodies directed against said microorganism of interest are added to the sample and the amount of bound secondary antibodies is measured.

10. The method according to claim 1 wherein the presence of biological material is detected by adding labelled secondary antibodies directed against to the sample and detecting bound secondary antibodies.

11. The method according to claim 9 wherein wherein the primary antibodies are labelled and signal generated from the primary antibody and secondary antibody is detected by optical imaging using an array of detectors.

12. The method according to claim 10 wherein wherein the primary antibodies are labelled and signal generated from the primary antibody and secondary antibody is detected by optical imaging using an array of detectors.

13. The method according to claim 9 wherein the signal generated from the primary antibody and secondary antibody is imaged by scanning the analyser using a single detector element.

14. The method according to claim 10 wherein the signal generated from the primary antibody and secondary antibody is imaged by scanning the analyser using a single detector element.

15. The method according to claim 9 wherein the signal generated from the primary antibody and secondary antibody is detected with a fixed (non-scanning) optical system by serially uncovering each cell in the analyser.

16. The method according to claim 10 wherein the signal generated from the primary antibody and secondary antibody is detected with a fixed (non-scanning) optical system by serially uncovering each cell in the analyser.

17. The method according to claim 9 wherein the signal generated from the primary antibody and secondary antibody is detected with a fixed (non-scanning) optical system by positioning of a single detector element in front of or behind each cell in the analyser.

18. The method according to claim 9 wherein the signal generated from the primary antibody and secondary antibody is detected electrically or electrochemically.

19. The method according to claim 9 wherein the proportion of available binding sites occupied is calculated from the ratio of the signals generated from the primary and secondary antibodies.
